(19) United States
(12) Patent Application Publication
(10) Pub. No.: US 2020/0197063 A1
(43) Pub. Date: Jun. 25, 2020

(54) Bone Filling Systems, Devices, and Methods of Use

(71) Applicant: Emory University, Atlanta, GA (US)

(72) Inventors: Janice Newsome, Atlanta, GA (US); Zachary Bercu, Atlanta, GA (US); Jonathan Martin, Atlanta, GA (US); Elizabeth Marr, Atlanta, GA (US); Nicholas Arce, Atlanta, GA (US); Kathryn Martin, Bonaire, GA (US)

(21) Appl. No.: 16/608,760

(22) PCT Filed: Apr. 25, 2018
(86) PCT No.: PCT/US18/29405

§ 371 Date: Oct. 25, 2019

Related U.S. Application Data
(60) Provisional application No. 62/489,976, filed on Apr. 25, 2017.

(51) Int. Cl. A61B 17/88

(52) U.S. Cl. CPC A61B 17/8811; A61B 2017/00469; A61B 17/8855; A61B 17/8808

(57) ABSTRACT

The systems, devices and methods can deliver a therapeutic material within a cavity of a joint while preventing leakage into the joint space. The systems may include a delivery device configured to deliver a therapeutic material to a treatment area within a bone cavity of a bone. The device may include a containment device. The containment device may include a body having a first end, a second, and a length there between, and an expandable member disposed at the first end of the body so as to cover the first end. The expandable member may be configured to move between an expanded state and a collapsed state. The system may further include an access device that includes a first channel and a second channel. The first channel may be configured to receive the expandable containment device and the second channel may be configured to receive the delivery device.

### CROSS-REFERENCE TO RELATED APPLICATIONS

This application claims the benefit of U.S. Provisional Application No. 62/489,976 filed Apr. 25, 2017. The entirety of this application is hereby incorporated by reference for all purposes.

### BACKGROUND

Bone conditions, such as avascular necrosis, osteoporosis, tumors, etc., can predispose the bone, as well as can cause the bone, to fracture or collapse. The treatment often includes repair and/or replacement of the one or more parts of the bone joint. For example, for a femoral head collapse, the treatment often is a total hip replacement. Hip replacements can be a highly invasive procedure that requires a long postoperative care and rehabilitation.

Procedures for repairing the bone, such as for the spine, may include applying a bone filler material to replace the degenerated tissue and/or to support or reinforce the bone. However, conventional devices may result in the leaking of the bone filling material into the joint space. This can result in a stabilization and complications (e.g., inflammatory response due to the leak, termination of nerves, etc.).

### SUMMARY

Thus, there is need for systems, devices and methods that can minimally invasively and accurately deliver a therapeutic material, such as a bone filling material, to a bone cavity to restore mechanical strength while being contained within the bone cavity.

The disclosure relates to systems, devices and methods that can deliver, minimally invasively, a therapeutic material within a cavity of a joint while preventing leakage.

In some embodiments, the systems may include a system for delivering a therapeutic material within a cavity of the bone. The system may include a delivery device configured to deliver a therapeutic material to a treatment area within a bone cavity of a bone. The system may further include a containment device including a body having a first end, a second, and a length there between, and an expandable member disposed at the first end of the body so as to cover the first end. In some embodiments, the expandable member may be configured to move between an expanded state and a collapsed state. The system may further include an access device having a first end, a second end, and a length there between. The access device may include a plurality of channels. The plurality of channels may include a first channel and a second channel. The first channel and the second channel may extend along the length of the access device. The first channel may be configured to receive the expandable containment device for deployment and the second channel may be configured to receive the delivery device for deployment.

In some embodiments, the expandable member may have a first surface, a second surface, and a fillable or expandable cavity disposed there between. The first surface and the second surface may have complimentary convex and/or concave shapes.

In some embodiments, when the expandable member is in the expanded state, the expandable member may be configured to engage an exterior surface of the bone.

In some embodiments, the delivery device may be configured to deliver the therapeutic material to the treatment area when the expandable member of the containment device is in the expanded state.

In some embodiments, the first channel may have a diameter that is smaller than a diameter of the second channel.

In some embodiments, the system may further include another access device having a first end, a second end, and a length there between. The other access device may include a channel disposed along the length from the first end to the second end. In some embodiments, the other access device may be configured to receive one or more boring devices.

In some embodiments, the access device may have a diameter along the length. The other access device may have a diameter along the length. The diameter of the other access device may be larger than a diameter of the access device. The channel of the other access device may be configured to receive the other access device.

In some embodiments, each of the access device and the other access device may include a handle member disposed about the second end. The handle members may be configured to mate when the access device is fully deployed within the other access device.

In some embodiments, the other access device may have a diameter at the first end that is larger than a diameter at the second end. The diameter of the other access device along the length may be tapered.

In some embodiments, the system may include one or more boring devices. Each boring device may include a first end, a second end, and a length there between. Each boring device may include a cutting edge disposed at the first end. The one or more boring devices may be configured to form a path through the bone cavity from a first (exterior) surface to a second (exterior) surface of the bone.

In some embodiments, the system may further include a void creation device configured to form a void within the bone cavity at the treatment area. The void creation device may be configured to compress or compact the tissue and/or excise the tissue within the bone cavity to form the void. In some embodiments, the void creation device may include an expandable member disposed at an end of a body. The expandable member may be configured to releasably expand and collapse. When the expandable member of the void creation device is expanded, the void creation device may be configured to compress or compact the tissue to form a void at the treatment area.

In some embodiments, the therapeutic material may be a bone filling material.

In some embodiments, the methods may include a method of stabilizing a bone by delivering a therapeutic material. In some embodiments, the method may include deploying an access device into an access path within a bone cavity of the bone from a first surface of the bone. The access device may have a first end, a second end, and a length. The access device may include a plurality of channels extending along the length from the first end to the second end. The access device may include deploying a containment device having an expandable member in a collapsed state through one of the channels of the access device and through the access path until the expandable member is outside of a second surface of the bone. In some embodiments, the first and second surfaces of the bone may correspond opposing ends of the access path. In some embodiments, the method may include expanding the expandable member so that the expandable member engages the second surface and covers an opening of the access path. The method may further include deploying a delivery device through another one of the channels of the access device. The method may further include delivering a therapeutic material to a treatment area while the expandable member is expanded. The first surface and second surface of the bone may correspond to an exterior proximal surface and an exterior interior surface, respectively.

In some embodiments, the expandable member may have a first surface, a second surface, and a fillable cavity disposed there between. In some embodiments, the first surface and the second surface may have complimentary convex and/or concave shapes.

In some embodiments, the channel may have a diameter that is smaller than a diameter of the other channel.

In some embodiments, the method may further include deploying another access device with a boring device mated with the other access device to form a first portion of the access path from the first surface of the bone to the treatment area. The method may further include removing the boring device and deploying the access device into the other access device. The access device may have a diameter that is smaller than a diameter of the other access device.

In some embodiments, the method may further include deploying another boring device through the access device to form a second portion of the access path from the treatment area to the second surface.

In some embodiments, the method may further include deploying another access device with a boring device mated with the other access device to form a portion of the access path from the first surface of the bone to the treatment area. The method may further include removing the boring device and deploying another boring device into the other access device. The method may also include removing the other access device and deploying the access device over the other boring device.

In some embodiments, the other access device may have a diameter at the first end that is larger than a diameter at the second end. The diameter of the other access device along the length may be tapered.

In some embodiments, each of the boring device and the other boring device may include a drill tip. The drill tip of the boring device and the other boring device may be different.

In some embodiments, the method may further include collapsing the expandable member and removing the containment device from the bone cavity through the access device. In some embodiments, the method may also include removing the delivery device through the other channel from the bone cavity and removing the access device from the bone cavity.

In some embodiments, the therapeutic material may be a bone filling material.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, emphasis being placed upon illustrating the principles of the disclosure.

FIG. 1A shows an example of an access device according to embodiments;

FIG. 1B shows a cross-sectional view of the device shown in FIG. 1A;

FIG. 2A shows an example of another access device according to embodiments;

FIG. 2B shows a cross-sectional view of the device shown in FIG. 2A;

FIG. 3 shows an example of a boring device according to embodiments;

FIG. 4A shows an example of another access device according to embodiments;

FIG. 4B shows a cross-sectional view of the device shown in FIG. 4A;

FIGS. 5A and 5B show examples of other cross-sectional views of the device shown in FIG. 4A according to embodiments;

FIG. 6 shows an example of another boring device according to embodiments;

FIG. 7A shows an example of a containment device having an expandable member in an expanded state, according to embodiments;

FIG. 7B shows an example of the containment device having the expandable member in a collapsed state, according to embodiments;

FIG. 7C shows an exploded view of the expandable member shown in FIG. 7A;

FIG. 8 shows an example of a therapeutic material delivery device according to embodiments;

FIG. 9A shows an example of a void creation device having an expandable member in an expanded state, according to embodiments;

FIG. 9B shows an example of the void creation device having the expandable member in a collapsed state, according to embodiments;

FIG. 10 shows an example of a method step to form a portion of an access path within a bone cavity using the boring device and the access device, according to embodiments;

FIG. 11 shows an example of the formed access path with the boring device and the access device deployed in the path, according to embodiments;

FIG. 12 shows an example of a method step to access the access path by deploying another access device, according to embodiments;

FIG. 13 shows an example of the formed access path with the access devices deployed, according to embodiments;

FIG. 14 shows an example of an optional method step to enlarge the treatment area by forming a void within the bone cavity using a void creation device, according to embodiments;

FIG. 15 shows an example of a method step to form another portion of the access path to an exterior surface of the bone cavity using the access device(s) and the boring device, according to embodiments;

FIG. 16 shows an example of the formed path with the access device(s) deployed, according to embodiments;

FIG. 17 shows an example of a method step to deploy and expand the expandable member of the containment device within the access device(s), according to embodiments;

FIG. 18 shows an example of a method step to deliver a therapeutic material to the treatment area by deploying the delivery device while the expandable member of the containment device is deployed, according to embodiments;

FIG. 19 shows an example of the bone that has been stabilized by the delivered therapeutic material, according to embodiments;

FIG. 20 shows an example of another method step to form an access path within a bone cavity using the boring device and the access device, according to embodiments;

FIG. 21 shows an example of a formed access path with the boring device and the access device deployed shown in FIG. 20, according to embodiments;

FIG. 22 shows an example of a method step to deploy another boring device in the bone cavity via the access device, according to embodiments;

FIG. 23 shows an example of the formed access path with the access and boring devices deployed shown in FIG. 22, according to embodiments;

FIG. 24 shows an example of a method step to use the boring device as a guide to remove the access device from the bone cavity, according to embodiments;

FIG. 25 shows an example of a method step to use the boring device as a guide to deploy another access device into the access path, according to embodiments;

FIG. 26 shows an example of a method step to form another portion of the access path to an exterior surface of the bone cavity using the access device and the boring device, according to embodiments; and

FIG. 27 shows an example of the access path formed within the bone cavity with the access device deployed, according to embodiments.

### DESCRIPTION OF THE EMBODIMENTS

In the following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

The systems, devices, and methods according to the disclosure can provide a minimally invasive (e.g., percutaneous) solution to repair a bone. The systems, devices, and methods include an expandable member that can engage (maintain a close interface) with an exterior surface of the bone cavity during the delivery of a therapeutic material, to thereby prevent leakage of the therapeutic material from the bone cavity into the (adjacent) joint space. Thereby, the systems, devices, and methods can provide a minimally invasive alternative to a joint replacement, with reduced risk of complications, reduced postoperative care, and reduced rehabilitation.

Although the embodiments and features described herein are specifically described for use in treating the femoral head (for example, femoral head collapse due to avascular necrosis), it will be understood that all of the devices, methods, and other features described below may be combined with one another in any suitable manner and may be adapted and applied for other treatments, for example, for weakened or fractured bone due to other traumatic or pathologic conditions, and/or to other regions and/or bone(s) (e.g., including but not limited to spine (vertebral body), humerus, talus, tibia, among others, or a combination thereof).

In addition, the words “proximal” and “distal” refer to directions closer to, and away from, respectively, an operator or clinician (e.g., surgeon, physician, nurse, technician, etc.) who would deploy or insert the devices described herein into the patient, with the tip-end (i.e., distal end) of the device inserted inside a patient's body. By way of example, the end of a device deployed or inserted inside the patient's body (e.g., the bone) would be the distal end of that device, while the end of the device outside the patient's body and/or closest to the clinician would be the proximal end of the device. A bone cavity or skeletal cavity may refer to an interior region of a bone or joint, such as interior of the vertebrae of the spine, the cancellous bone of regions close to joints, e.g. knee and hip, etc.

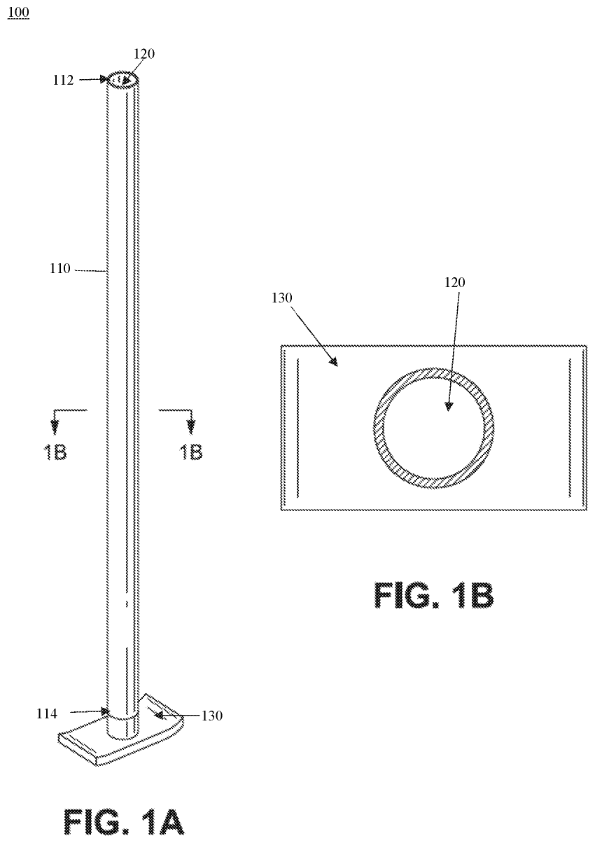

FIGS. 1A-9B show examples of devices of a system according to embodiments. In use, a combination of the devices of the system can be deployed in a purposeful manner along an access path formed in the bone cavity to deliver a therapeutic material (e.g., bone filling material) to a treatment area within the bone cavity, while being contained by a containment device (e.g., containment device 700).

In some embodiments, the access path (also referred to as “the path”) may refer to a path formed in the bone cavity of a bone from an opening formed in a proximal surface of the bone to an opening formed in the distal surface of the bone. In some embodiments, the path may be formed by deploying one or more boring devices, e.g., via one or more access devices. In some embodiments, the access path may be formed in one or more portions using different boring devices with one or more access devices. For example, the access path may be formed as a first portion and a second portion using at least two different boring devices that may be deployed with one or more access devices. The first portion may include the opening formed within the proximal surface of the bone to about the treatment area within the cavity, and that second portion may include the treatment area to the opening formed within the distal surface of the bone.

It will be understood that a system may include any combination of the devices, features of the devices, additional and/or alternative of the devices described herein. It will be understood that a combination of one or more of the devices shown in FIG. 1A-9B can be part of a prepackaged kit.

FIGS. 1A and 1B show an example of an access device 100 according to some embodiments. The access device 100 that can be used alone and/or used with one or more boring devices, which can form one or more portions of an access path within the bone cavity.

In some embodiments, the access device 100 may be designed to extend from an internal body part in a subject to outside of the subject's body. As shown in FIG. 1A, the access device 100 may include a body 110 having a first (or distal) end 112 (to be position in or near the bone to be treated), an opposing second (or proximal) end 114 (to be accessible to a user (e.g., clinician)), and length there between. The first end 112 and the second end 114 may each include an opening. The access device 100 may include one or more channels that extend along the length of the body 110 from the first end 112 to the second end 114. As shown in the FIG. 1A, the access device 100 may include at least one channel 120 disposed along the length of the body 110 from the first end 112 to the second end 114. The opening at each of the ends 112 and 114 may correspond to the channel 120.

In some embodiments, the access device 100 may include a handle 130 disposed at the second end 114. The handle 130 may include at least an opening corresponding to the channel 120. In some embodiments, the handle 130 may have tapered flanges extending from the opening to the channel 120. In some embodiments, the access device 100 may have a different handle.

As shown in these figures, the access device 100 may have the same diameter along the length from the first end 112 to the second end 114, as shown in FIG. 1B. In some embodiments, the access device 100 may have a tapered diameter.

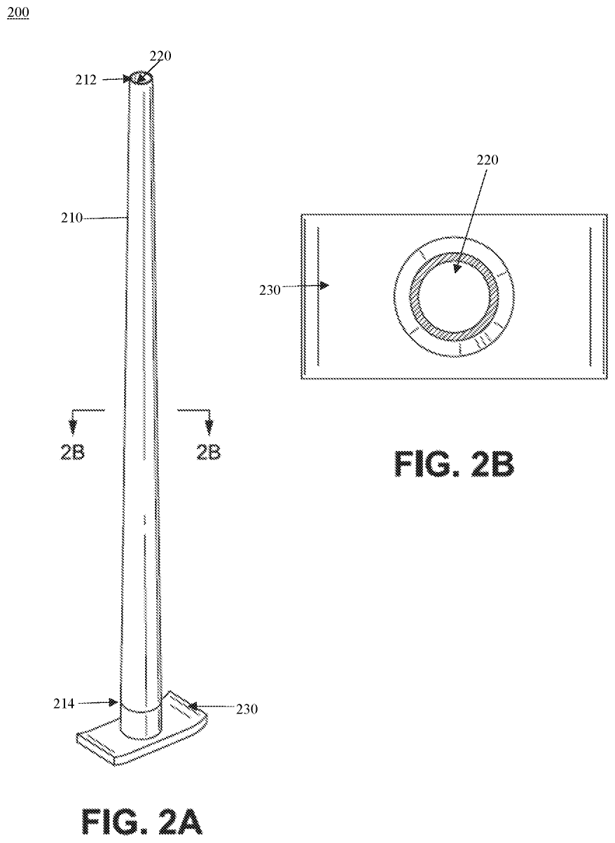

FIGS. 2A and 2B show an example of an access device 200 having a tapered diameter. Like the access device 100, the access device 200 may be used with one or more drills or boring devices to form one or more portions of the access path within the bone cavity.

As shown in FIG. 2A, the access device 200 may include a body 210 having a first (or distal) end 212, an opposing second (or proximal) end 214, and length there between. The first end 212 and the second 214 may each including an opening. The access device 200 may include one or more channels that extend along the length from the first end 212 to the second end 214. As shown in the FIG. 2A, the access device 200 may include at least one channel 220 disposed along the length of the body 210 from the first end 212 to the second end 214. The opening at each of the ends 212 and 214 may correspond to the channel 220.

In some embodiments, the access device 200 may include a handle 230 disposed at the second end 214 of the body 210. The handle 230 may include at least one opening corresponding to the channel 220. In some embodiments, the handle 230 may have tapered flanges extending from the opening to the channel 220. In some embodiments, the access device 200 may have a different handle.

As shown in FIGS. 2A and 2B, the diameter of the access body 210 and the channel 220 may taper from smaller to larger from the first end 212 to the second end 214. In some uses, the tapered diameter may provide a more accurate placement of the one or more boring devices and/or formation of the access path.

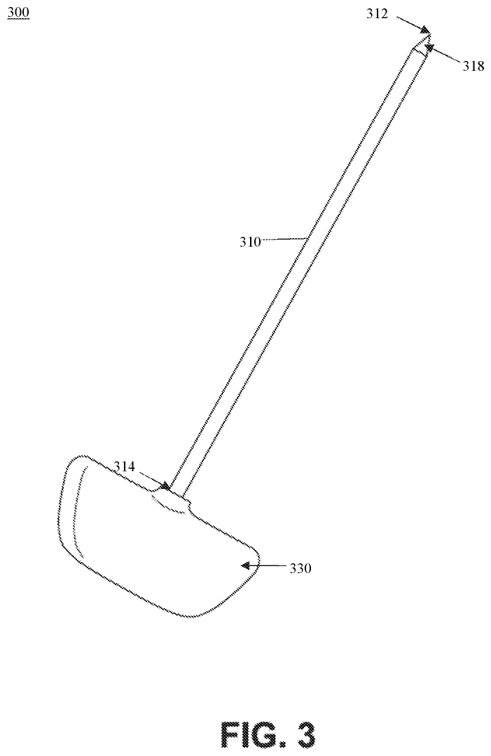

FIG. 3 shows an example of a boring device 300 according to some embodiments. In some embodiments, the boring device 300 may include a body 310 having a first (or distal) end 312, an opposing second (or proximal) end 314, and length there between. The body 310 of the boring device 300 may be a drill bit. In some embodiments, the boring device 300 may include a diamond shaped cutting edge (e.g., drill tip) 318 disposed at the first end 312. In other embodiments, the boring device 300 may have a different cutting edge or drill tip.

In some embodiments, the boring device 300 may include a handle 330 that can be removably disposed at the second end 314. In some embodiments, the handle 330 may be complimentary to the handle of one or more access devices (e.g., access devices 100 and/or 200) so that the handles of the boring device 300 and the one or more access device(s) (e.g., access devices 100 and/or 200) can mate when inserted or deployed together and/or separately into the bone cavity. In some embodiments, the handle 330 of the boring device 300 may also be configured to receive other device(s), such as a mallet device, a tamp device, among others, or a combination thereof.

In some embodiments, the system may include additional and/or alternative boring devices (e.g., drill bits). In some embodiments, the system may include any number of boring devices (e.g., drill bits) of the same and/or different size (e.g., diameter, length, etc.), drill tip, shape, among others, or a combination thereof.

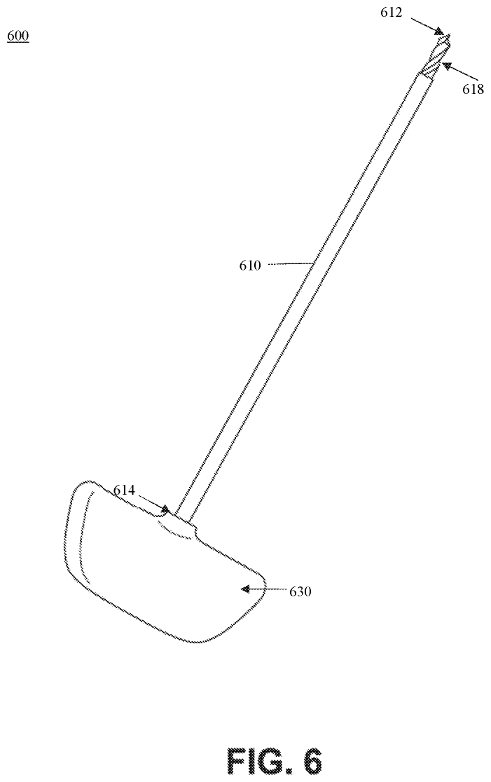

FIG. 6 shows an example of a boring device 600 according to some embodiments. Like the boring device 300, the boring device 600 may include a body 610 having a first (or distal) end 612, an opposing second (or proximal) end 614, and length there between. The body 610 of the boring device 600 may be a drill bit. In some embodiments, the boring device 600 may include a cutting edge (drill tip) 618 disposed at and near the first end 612. The cutting edge 618 may include a corkscrew shape and/or pattern.

In some embodiments, the boring device 600 may include a handle 630 that can be removably disposed at the second end 614. In some embodiments, the handle 630 may be complimentary to the handle of one or more access device(s) (e.g., devices 100, 200 and/or 400) so that the handles of the boring device 600 and the access device(s) mate (e.g., devices 100, 200 and/or 400) when inserted or deployed together and/or separately into the bone cavity. In some embodiments, the handle 630 of the boring device 600 may also be configured to receive other device(s), such as a mallet device, a tamp device, among others, or a combination thereof.

In some embodiments, the handle 630 and the handle 330 may be the same or different. For example, if the system includes at least two boring devices 300 and 600, the system may include one handle that can be used with both of the bodies 310 and 610.

In some embodiments, the path may be formed in one or more portions using different boring devices and/or access devices. For example, the boring device 300 may be configured to be deployed with one or more access devices to form the first portion of the access path, including a hole within the proximal surface of the bone, and the boring device 600 may be configured to be deployed with one or more access devices to form the second portion of the path, including a hole within the distal surface of the bone. In some embodiments, the path may be formed using one or more different access devices, one or more different boring devices, a different combination of access device(s) and/or boring device(s), among other things, or a combination thereof.

In some embodiments, the body 610 may have different dimensions than the body 310. For example, the body 610 may have a smaller diameter than the body 310, a length that is longer than the body 310, among others, or a combination thereof.

In some embodiments, one or more access devices may be configured to provide access to the (targeted) treatment area within the bone cavity and/or to deliver the therapeutic material to the treatment area. In some embodiments, the system may additionally or alternatively include an access device having more than one channel. At least one of the channels may be configured to receive a device having an expandable member configured to establish a barrier on a distal side or surface of the bone (at the end of the path) and at least another one of the channels may be configured to receive a device configured to deliver the therapeutic material to the treatment area while the expandable member is expanded.

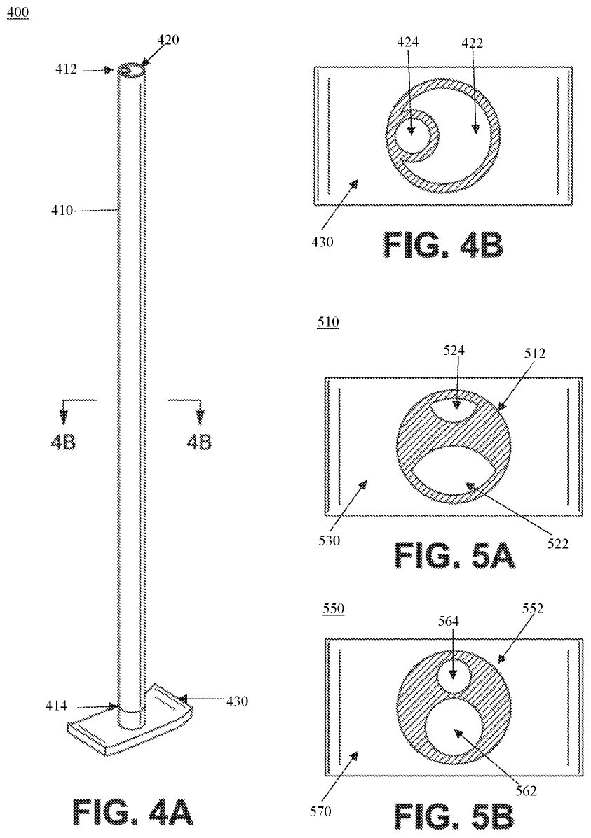

FIGS. 4A and 4B show an example of an access device 400 according to some embodiments. The device 400 may be configured to guide the deployment of one or more boring devices to form at least a portion of the path, to guide a deployment of a containment device having an expandable member that engages the distal side/surface of the bone to seal the path when expanded, to guide a deployment of a delivery device to deliver the therapeutic material into the treatment area while the expandable member is deployed, among others, or a combination thereof.

In some embodiments, the device 400 may be designed to extend from an internal body part in a subject to outside of the subject's body. In some embodiments, the device 400 may have a smaller outer diameter than the device 100 and may be configured to be inserted or deployed within the device 100.

As shown in FIG. 4A, the device 400 may include a body 410 having a first (or distal) end 412, an opposing second (or proximal) end 414, and length there between. The access device 400 may include more than one channel 420 that extends along the length of the body 410 from the first end 412 to the second end 414. As shown in the FIG. 4A, the device 400 may include at least two channels disposed along the length of the body 410 from the first end 412 to the second end 414. The access device 400 may include a handle 430 disposed at the second end 414. The handle 430 may include at least an opening corresponding to each channel 420.

In some embodiments, the channels 420 may include at least a first channel and a second channel. The first channel may have a larger diameter than a second channel. By way of example, the larger first channel may be configured to receive and be a guide for devices/instruments (e.g., devices 800, 900, etc.) that are to be deployed within bone cavity and the smaller second channel may be configured to receive and be a guide for devices/instruments (e.g., devices 600, 700, etc.) that are to be deployed outside of the bone cavity (e.g., beyond the proximal surface).

FIG. 4B shows a cross-section of the device 400. For example, the channels 420 may include a first channel 422 that has a larger diameter than a second channel 424. In this example, the first channel 422 may surround the second channel 424. The second channel 424 may have a circular shaped cross-section and the first channel 422 may have a half moon cross-section.

In some embodiments, the channels 420 may have a different cross-section. The cross-section of the channels 420 may have any shape. FIGS. 5A and 5B show examples 510 and 550 of different cross-sections of the body 512 and 552 with respect to the handles 530 and 570, respectively. In these examples, the bodies 512 and 552 and the respective handles 530 and 570 may have a similar appearance to the device 400 but for the cross-sections shown. It will be understood that the cross-section of the channels 420 are not limited to these examples and that the cross-section of the channels 420 may be different from those shown in the figures.

As shown in FIG. 5A, the body 512 may include a first channel 522 and a second channel 524 that is smaller than the first channel 522. The first channel 522 and the second channel 524 may have substantially the same shape but be different in size. The first channel 522 and the second channel 524 may each have a convex or concave cross-section shape.

As shown in FIG. 5B, the body 552 may include a first channel 562 and a second channel 564 that is smaller than the first channel 562. The first channel 562 and the second channel 564 may have substantially the same shape but be different in size. The first channel 562 and the second channel 564 may each have a circular cross-section shape.

In some embodiments, the channels 420 may include more than two channels. The channels 420 may include any number of channels. For example, the channels 420 may include three or four channels. For example, the channels 420 may include two or more channels configured to deliver the therapeutic materials.

In some embodiments, the body 410 may have a solid outer surface as shown. In some embodiments, the body 410 may include one or more openings disposed along the length of the body 410 to communicate with one or more of the channels 420.

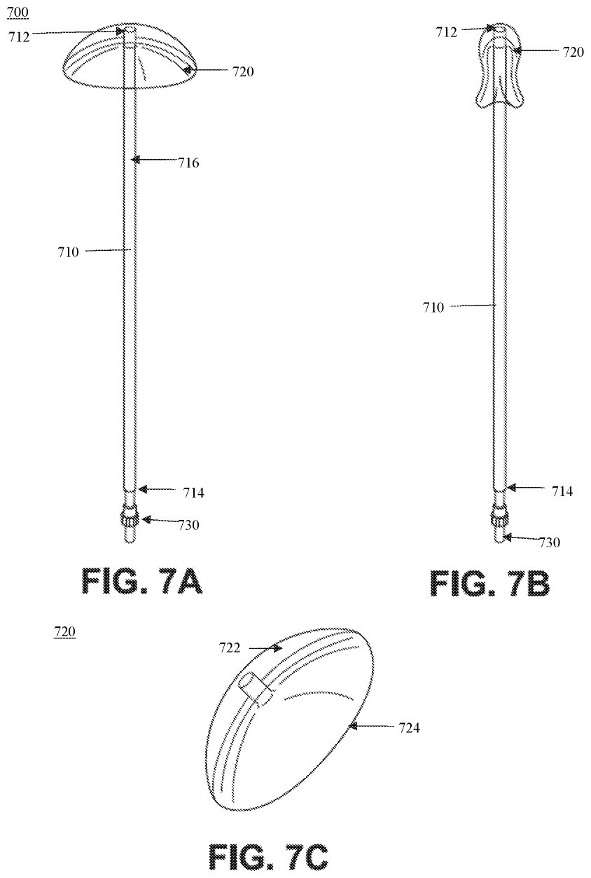

FIGS. 7A-C show an example of an expandable (containment) device 700 including an expandable member 720 configured to releasably expand to cover or seal the distal surface of the bone (e.g., distal end of the path), according to embodiments. This way, the expandable member 720 may act as a barrier to contain the therapeutic material within the treatment area (and the bone cavity), thereby preventing leakage of the therapeutic material from the bone cavity into the (adjacent) joint space during and/or after delivery of the therapeutic material.

As shown in FIGS. 7A and 7B, the device 700 may include a body 710 having a first (or distal) end 712, an opposing second (or proximal) end 714, and length there between. The body 710 may include one or more channels 716. In some embodiments, the device 700 may include an expandable member 720 disposed about the first end 712 and releasably configured to move between an expanded state and a collapsed state. As shown in the FIGS. 7A and 7C, when the expandable member 720 is in the expanded or inflated state, the expandable member 720 may be configured to expand about the first end 712 so as to surround the first end 712. As shown in the FIG. 7B, when the expandable member 720 is in the collapsed or deflated state, the expandable member 720 may be configured to be deployed through one or more access devices to the exterior, distal side/surface of the bone (via the formed access path).

In some embodiments, the device 700 may include an adapter or handle 730 disposed at the second end 714, for example, to directly or indirectly (through another port) connect to one or more devices configured to expand and/or collapse the expandable member 720. The device 700 may include a different adapter and/or handle.

In some embodiments, the expandable member 720 may be configured to expand/inflate using fluid(s), material(s), among others, or a combination thereof. The expandable member 720 may be a balloon. In some embodiments, the expandable member 720 may be made of any compliant material(s), such as poly(ethylene terephthalate) (PET), nylon, among others, or a combination thereof.

In some embodiments, as shown in FIG. 7C, the expandable member 720 may include a first surface 722, an opposing second surface 724, and a fillable/expandable cavity there between. The expandable member 720 may be in communication with the one or more channels 716 to receive and/or remove fluid(s) and/or material(s) (via the adapter 730) to respectively expand and/or deflate. When the expandable member 720 is expanded, the second surface 724 may be configured to engage the exterior, distal surface of the bone. For example, the expandable member 720 may be configured to engage the exterior surface of the femoral head.

In some embodiments, the expandable member 720 may have any shape, including oval, oblong, triangular, square, polygonal, irregular, uniform, non-uniform, offset, staggered, tapered, among others, or a combination thereof. In some embodiments, the surfaces 722 and 724 may have a complimentary shape. In some embodiments, the first surface 722 and second surface 724 may have complimentary convex and concave shapes, as shown in FIG. 7C. In some embodiments, the first surface 722 and the second surface 724 may have a different shape that may or may not be complimentary. The shape may depend on a particular anatomy of the bone to be treated.

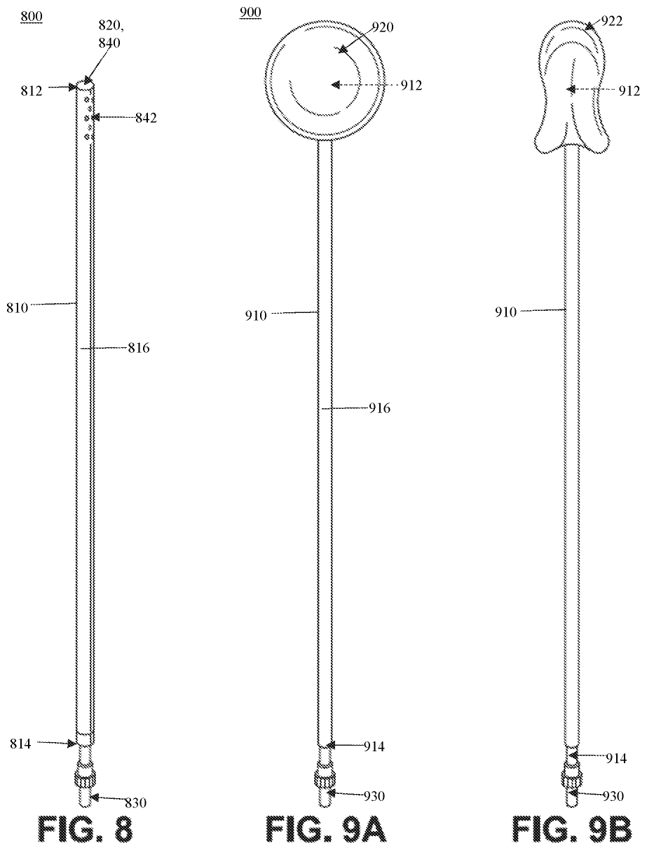

FIG. 8 show an example of a therapeutic material delivery device 800 according to embodiments. In some embodiments, the device 800 may be designed to extend from an internal body part in a subject to outside of the subject's body. The device 800 may include a body 810 having a first (or distal) end 812, an opposing second (or proximal) end 814, and length there between. The first end 810 and the second end 812 may each including an opening. The delivery device 800 may include one or more channels 816 that extend along the body 810. The device 800 may include at least one channel 820 disposed along the length of the body 810 from the first end 812 to the second end 814. The opening at each end 812, 814 may correspond to the channel 820.

In some embodiments, the device 800 may include one or more openings configured to deliver the therapeutic material(s) to the treatment area. In some embodiments, the one or more openings may include the opening 840 disposed about the first end 812; one or more additional openings 842 disposed along the length of the body 810, among others, or a combination thereof. For example, the one or more openings 842 may be disposed in a pattern disposed along one or more sides of the body 810 near the first end 812.

In some embodiments, the device 800 may include an adapter or handle 830 disposed at the second end 814, for example, to directly or indirectly (through another port) connect to one or more devices (e.g., syringe, other injection devices, etc.) configured to cause the delivery of the therapeutic material. The device 800 may include a different adapter and/or handle.

In some embodiments, the therapeutic material(s) may include a bone filling material, a bone growth promoting material, a therapeutic drug, a tissue graft, a population of cells, a biological matrix, any other injectable material, among others, or a combination thereof. As used herein, a bone filler material many include any material or combination of materials that may be used for the treatment of bone, such as, for example, autograft, allograft, demineralized bone matrix, mineral composites, blocks, granules and pellets and bone cement, such as, for example, polymethylmethacrylate (PMMA)-based material (e.g., Kyphon HV-R, ActivOs, ActivOs 10, Xpede, etc.), calcium phosphate (e.g., Skaffold, Norian, Hydroset, KyphOs FS, etc.), calcium sulfate (e.g., OsteoSet), elastomeric polymer, among other injectables, or a combination thereof.

In some embodiments, the system may include one or more additional devices configured to form and/or enlarge a void within the bone cavity at the targeted treatment area and/or along the path. For example, the one or more additional devices may be configured to form and/or enlarge the treatment area or path by applying force to compress or compact, excising to remove tissue (e.g., cancellous bone, bone marrow, etc.) from the bone cavity, among others, or a combination thereof. By way of example, the one or more additional devices may include an expandable member configured to engage the tissue, for example, by applying force to the tissue within the bone cavity; one or more excising devices (not shown) configured to excise the tissue within the bone cavity; among others; or a combination thereof.

FIGS. 9A and 9B show an example of a device 900 that includes an expandable member 920 that is configured to compress or compact tissue (e.g., cancellous bone, bone marrow, etc.) within the bone cavity. As shown in FIGS. 9A and 9B, the device 900 may include a body 910 having a first (or distal) end 912, an opposing second (or proximal) end 914, and length there between. The body 910 may include at least one or more channels 916 that extend along the body 910. The expandable member 920 may be disposed about the first end 912 and releasably configured to move between an expanded state and a collapsed state. As shown in the FIG. 9A, when the expandable member 920 is in the expanded or inflated state, the expandable member 920 may be configured to expand about the first end 912. As shown in the FIG. 9B, when the expandable member 920 is in the collapsed or deflated state, the expandable member 920 may be configured to be deployed through one or more access devices to within the cavity to form and/or enlarge a void (e.g., at the treatment area) and/or the path.

In some embodiments, the device 900 may include an adapter or handle 930 disposed at the second end 914, for example, to directly or indirectly (through another port) connect to one or more devices configured to expand and/or collapse the expandable member 920. The device 900 may include a different adapter and/or handle.

In some embodiments, the expandable member 920 may be configured to expand/inflate using fluid(s), material(s), among others, or a combination thereof. The expandable member may be a balloon. The expandable member 920 may be in communication with the one or more channels 916 to receive and/or remove fluid(s) and/or material(s) (via the adapter 930) to respectively expand and/or deflate. In some embodiments, the expandable member 920 may be made of any compliant material(s), such as poly(ethylene terephthalate) (PET), nylon, among others, or a combination thereof.

In some embodiments, the expandable member 920 may have a circular shape when expanded/inflated. In this example, the expandable member 920 may include an outer surface 922 configured to engage and apply force to tissue (e.g., cancellous bone, bone marrow, etc.) within the cavity when the expandable member 920 is expanded.

The expandable member 920 may have any shape, including oval, oblong, triangular, square, polygonal, tapered, among others or a combination thereof. The shape may depend on a particular anatomy of the bone to be treated.

In some embodiments, the system may include one or more of additional and/or alternative devices. For example, the system may include biopsy device(s), other device(s) to compact and/or remove tissue from the bone cavity, boring device(s), access device(s), among others, or any combination thereof.

In some embodiments, the components/devices of the system can be made of radiolucent materials such as polymers. Radiomarkers may be included for identification under x-ray, fluoroscopy, CT or other imaging techniques.

In some embodiments, the handles and/or adapters described herein may act to control the depth of insertion or deployment of the devices of the system, as well as to control the depth of insertion of other device(s) of the system are placed within the bone cavity. In other embodiments, the system may include a member, such as a removable spacer clip, for example, that can disposed on the body to surround the access device 100.

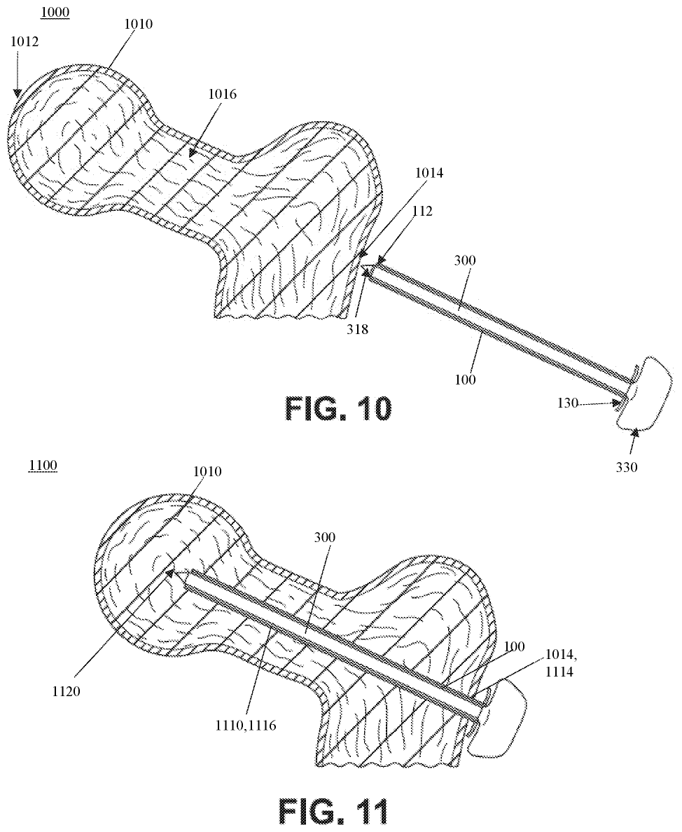

FIGS. 10-27 illustrates an examples of a procedure with regards to treat a femoral head using the system according to embodiments. FIGS. 10-15 and 20-27 show methods of forming an access path using the system described therein. FIGS. 15-19 show methods of delivering a therapeutic material to a targeted treatment area disposed along the path within the bone cavity. It will be understood that (i) the path may be formed and/or (ii) the path and/or treatment area (e.g., void) may be enlarged, using different devices and/or systems. It will also be understood that the methods and/or systems are not limited to use with respect to the femoral head and may be used with other bones/joints (e.g., tibia, spine, ankle, etc.). During the steps, the use of microsurgical and image guided technologies may be employed to access, view and repair bone deterioration or damage.

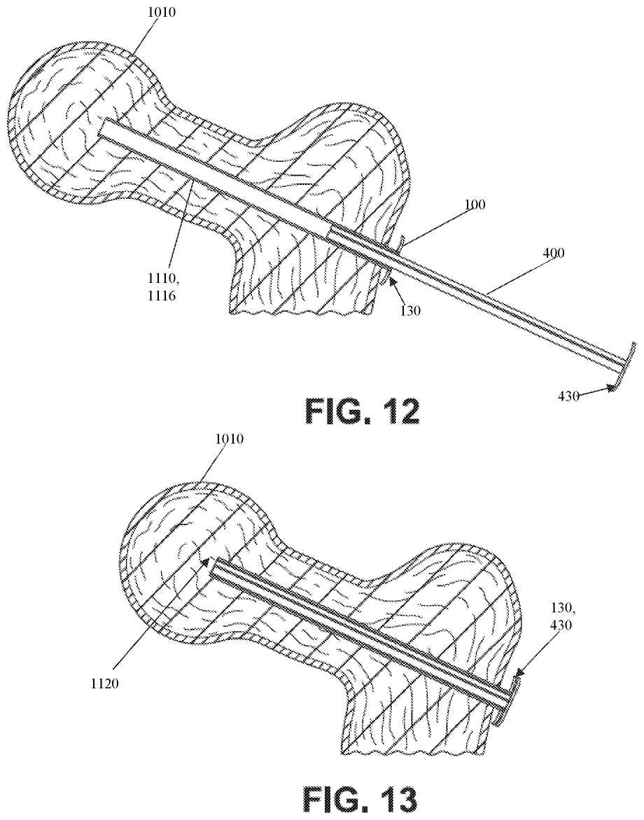

FIGS. 10 and 11 show an example of establishing or forming (a first portion of) an access path 1110 within a bone cavity 1016 of a femoral head 1010. As shown in the FIG. 10, the access device 100 with the boring device 300 may be deployed into the bone cavity 1016 starting at a (proximal) surface 1014 of the femoral head 1010. For example, at time of deployment, the handle 130 of the access device 110 and the handle 330 of the boring device 300 may be mated so that the cutting edge 318 extends and protrudes from the first end 112. This way, the boring device 330 may be used as a drill to form a portion of the access path 1110.

FIG. 11 shows the boring device 300 and the access device 100 deployed within the bone cavity 1016. In some embodiments, the access device 100 with the boring device 300 may form the opening 1114 at the surface 1014 and the first portion 1116 of the path 1110. The access device 100 and the boring device 300 may be deployed so that the first ends are deployed within the cavity 1016 to about the (targeted) treatment area 1120.

After the access device 100 is deployed, the boring device 300 may be removed from the access device 100. In some embodiments, the access device 400 may then be deployed into the access path 1110 via the access device 100 as shown in FIG. 12. For example, as shown in FIGS. 12 and 13, the access device 400 may be deployed into the access device 100 until the handles 430 and 130 mate. As shown in FIG. 13, the first ends 112 and 412 of the access devices 100 and 400 may be substantially aligned when the access device 400 is deployed in the access device 100 so that the handles 430 and 130 mate.

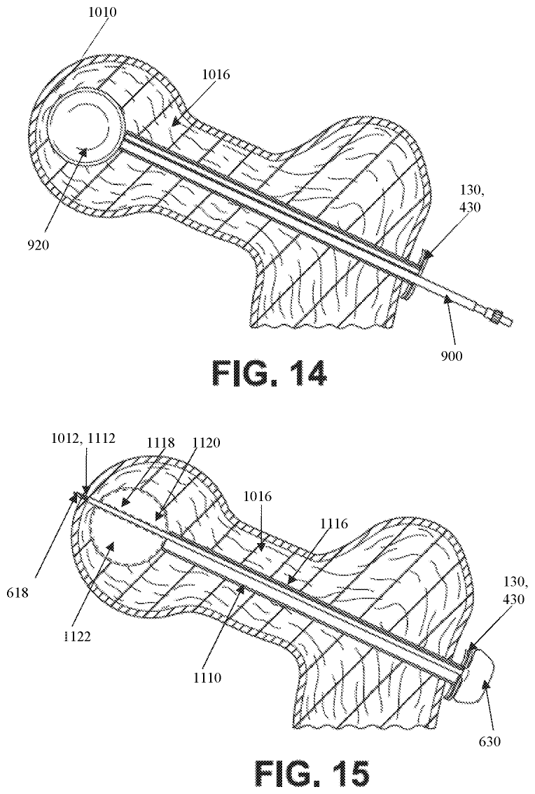

In some embodiments, the treatment area 1120 and/or (any portion of) the path 1110 may be enlarged, for example, by forming a void, using one or more devices. For example, as shown in FIG. 14, a void 1122 may be formed at the treatment area 1120 by deploying the device 900 into the one of the channels of the access device 400 in a collapsed state until the expandable member 920 is past the first end 412. By way of example, the device 900 may be deployed through the first channel 422 into the bone cavity 1016. After the device 900 is properly positioned, the expandable member 920 may be expanded via the adapter 930, for example, connected to a fluid and/or material delivery device. The expansion of the expandable member 920 may cause the tissue to compress or compact thereby forming the void 1122 within the bone cavity 1016 at the treatment area 1120. The expandable member 920 may then be collapsed and the device 900 may be removed from at least the access device 400.

In some embodiments, the void 1122 may be formed with another device, such as an excising device. In some embodiments, the method may omit the step of forming the void 1122, for example as shown in FIG. 14.

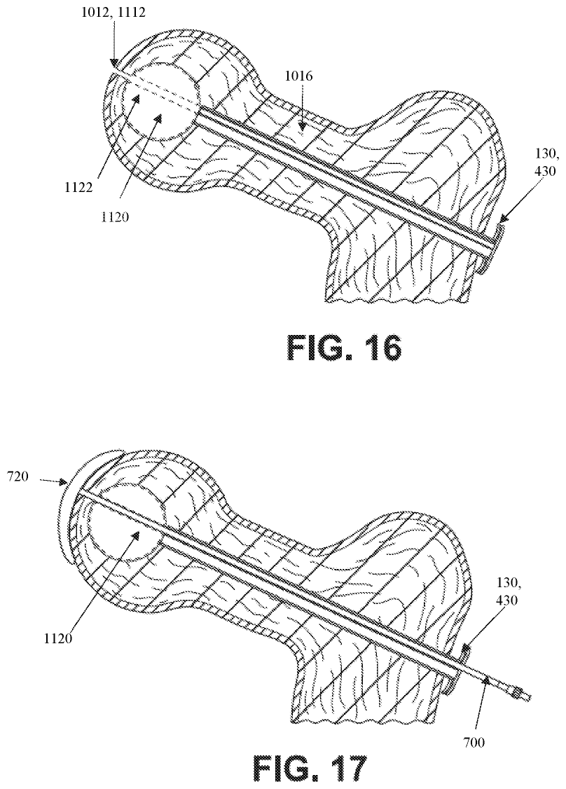

In some embodiments, the method may include deploying the boring device 600 through the cavity 1016 to form the second portion 1118 of the access path 1110 to and including an opening 1112 formed at the outside or distal surface 1012 of the femoral head 1010. For example, as shown in FIG. 15, the boring device 600 may be deployed, via at least the access device 400, into and through the cavity 1016 until the handles 630 and 430 mate, for example, as shown in FIG. 15. The boring device 600 may form the second portion 1118 of the path 1110 and the opening 1112 at the surface 1012. The boring device 600 may then be removed from the cavity 1016, for example, via at least the access device 400. As shown in FIG. 16, the boring device 600 may be used to form the second portion 1118 of the path 1110 so that the path 1110, including the treatment area 1120, now extends from the opening 1114 at the surface 1014 to the opening 1112 at the surface 1012 of the femoral head 1010.

After the path 1110 is formed from the opening 1114 at the surface 1014 through the cavity 1016 to the opening 1112 on the surface 1012 of the femoral head 1010, the method may further include deploying the containment device 700 in a collapsed state through one of the channels of the access device 400 and the access path 1110 through the opening 1112. Once the expandable member 720 is outside or exterior to the surface 1012 and through the opening 1112, the expandable member 720 may be expanded, for example, by delivering material(s) and/or fluid(s) to the expandable member 720 via the adapter 730, so that the expandable member 720 engages the surface 1012 and seals the opening 1112 at the surface 1012, as shown in FIG. 17. For example, the containment device 700 may be deployed via the channel 424 of the device 400. The second surface 724 of the containment device 700 may directly engage the surface 1012.

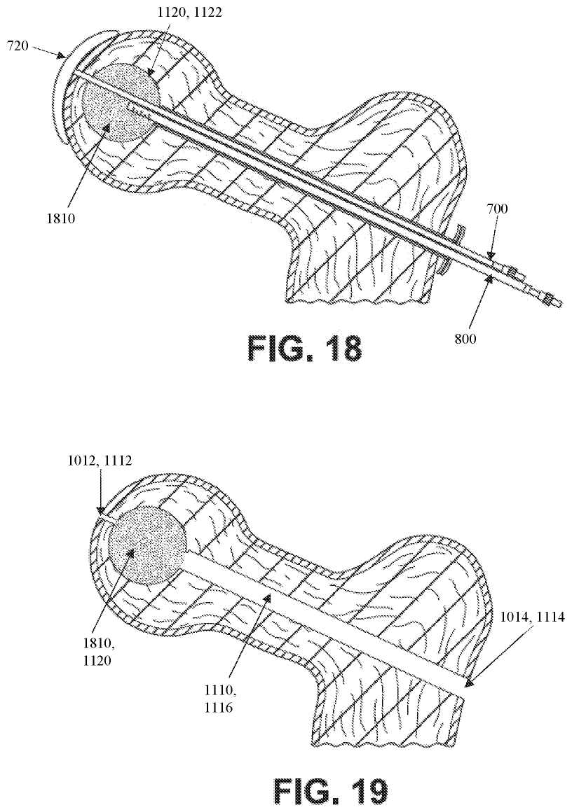

While the expandable member 720 of the containment device 700 is deployed, the delivery device 800 may be deployed to the treatment area 1120 via another one of the channels of the device 400 so that the one or more delivery openings 840 and/or 842 are past at least the first end 412 of the access device 400 and into the treatment area 1120. For example, the delivery device 800 may be deployed via the channel 422 of the device 400. Once properly positioned, the delivery device 800 can deliver a therapeutic material 1810 into the treatment area 1120 while the expandable member 720 is expanded so that the second surface 724 engages the surface 1012, as shown in FIG. 18.

Upon completion of the procedure, the surgical instruments and assemblies can be removed. As shown in the FIG. 19, the therapeutic material 1810 can remain in the treatment area 1120. The expandable member 720 can prevent leakage of the therapeutic material 1810 from the bone cavity 1016 into the (adjacent) joint/bone space. In some embodiments, the opening 1114 formed the bone and/or remaining portion of the path 1110 may be filled with the therapeutic material or different material (e.g., a bone cement) to provide further support for the repaired bone, and the incision may be closed.

Although FIGS. 17-19 show the delivery of the therapeutic material with both the access devices 100 and 400 deployed, it will be understood that the steps shown in FIGS. 17-19 may be performed using the access device 400 without the access device 100 (e.g., for example, using the deployment configuration shown in FIG. 27), with other access device(s), among others, or a combination thereof.

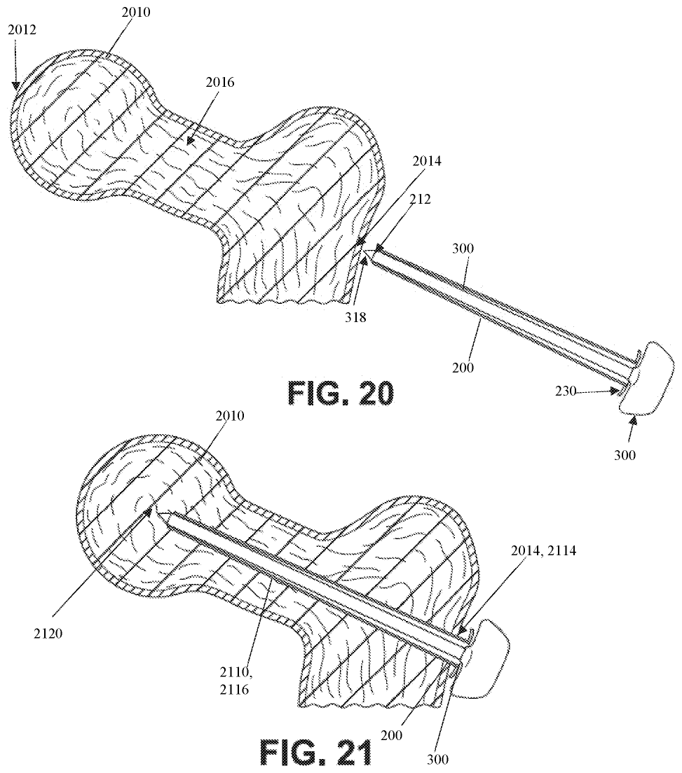

In some embodiments, the access path in the bone cavity for the access device 400 may be formed using alternative access device(s). By way of example, FIG. 20-27 show an alternative method steps (e.g., to steps FIGS. 10-16) for forming an access path in the bone cavity 1016 for the access device 400.

FIGS. 20 and 21 show an example of establishing or forming a first portion 2116 of an access path 2110 within the bone cavity 2016 of a femoral head 2010. As shown in the FIG. 20, the access device 200 with the boring device 300 may be deployed into the bone cavity 2016 starting at a surface 2014 of the femoral head 2010. For example, at time of deployment, the handle 230 of the access device 200 and the handle 330 of the boring device 300 may be mated so that the cutting edge 318 extends and protrudes from the first end 212. This way, the boring device 330 may be used as a drill to form the access path 2110.

FIG. 21 shows the boring device 300 and the access device 200 deployed within the bone cavity. In some embodiments, the access device 200 with the boring device 300 may form the opening 2114 at the surface 2014 and the first portion 2116 of the path 2110. The access device 200 and the boring device 300 may be deployed so that the first ends are deployed within the cavity 2016 to about the (targeted) treatment area 2120.

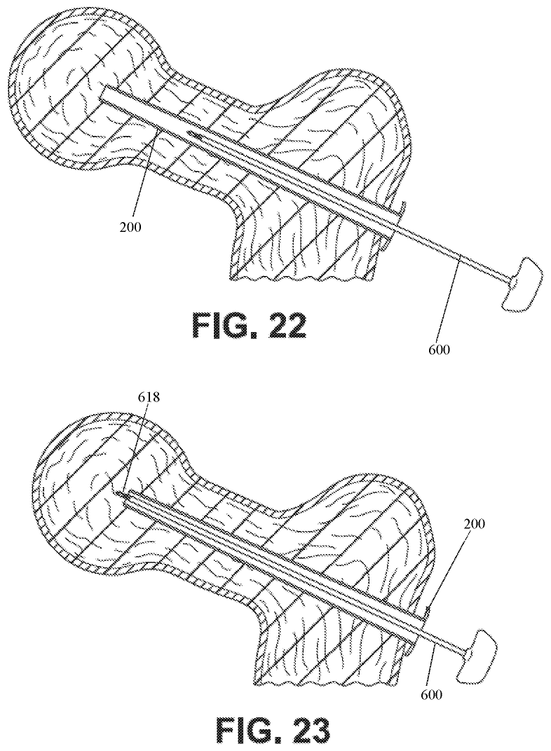

After the access device 200 is deployed to this position shown in FIG. 21, the boring device 300 may be removed from the access device 200. In some embodiments, the boring device 600 may then be deployed into the access path 2110 via the access device 200, as shown in FIG. 22. For example, as shown in FIG. 23, the boring device 600 may be deployed into the access device 200 until the cutting edge 618 is past the first end 212 of the access device 200.

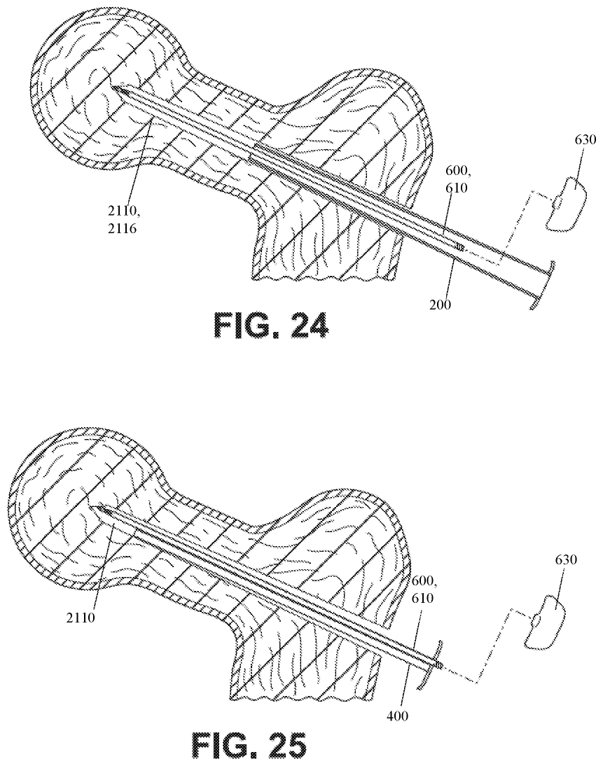

After the boring device 600 is properly positioned with respect to the treatment area 2120 within the bone cavity 2016, the handle 630 may be removed so that the access device 200 may be removed from the bone cavity 2016 over the body 610, as shown in FIG. 24.

In some embodiments, the method may include deploying the access device 400 into a portion of the path 2110 so that one of the channels of the access device 400 are over the body 610 of the boring device 600, as shown in FIG. 25. For example, the access device 400 may be deployed so that the body 610 of the boring device 600 goes through the channel 424 of the access device 400.

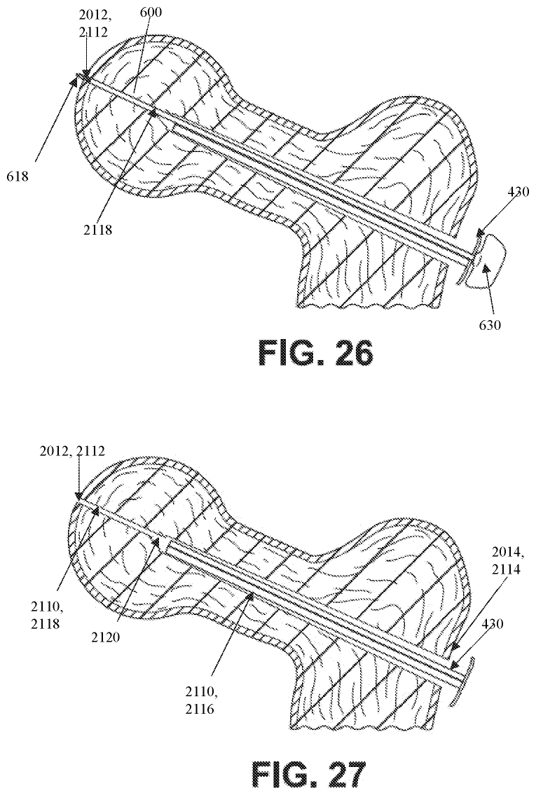

After the access device 400 is deployed into the cavity 2016 over the boring device body 610, the method may include deploying the boring device 600 further into the cavity 2016 to form the second portion 2118 of the access path 2110 to and including the opening 2112 at an outside or distal surface 2012 of the femoral head 2016, as shown in FIG. 26. For example, as shown in FIG. 26, the boring device 600 may be deployed into and through the cavity 2016 until the handles 630 and 430 mate. The boring device 600 may then be removed from the cavity 2016 via the access device 400. As shown in FIG. 27, this results in a formation of the path 2110 within the cavity 2016 from the opening 2114 at the proximal surface 2014 to the opening 2112 at the distal surface 2012 and the access device 400 being deployed within the path 2110. Next, the therapeutic material may be delivered to the treatment area 2120 disposed along the path 2110 using the devices 700 and 800 deployed via the access device 400, using the steps described with respect to and shown in FIGS. 17-19 for the treatment area 1120 disposed along the path 1110.

While the disclosure has been described in detail with reference to exemplary embodiments, those skilled in the art will appreciate that various modifications and substitutions may be made thereto without departing from the spirit and scope of the disclosure as set forth in the appended claims. For example, elements and/or features of different exemplary embodiments may be combined with each other and/or substituted for each other within the scope of this disclosure and appended claims.

1. A system for delivering a therapeutic material within a cavity of a bone, comprising:
a delivery device configured to deliver a therapeutic material to a treatment area within a bone cavity;
a containment device including a body having a first end, a second, and a length there between, and an expandable member disposed at the first end of the body so as to cover the first end;
the expandable member being configured to move between an expanded state and a collapsed state; and
an access device having a first end, a second end, and a length there between;
the access device including a plurality of channels, the plurality of channels including a first channel and a second channel; and
the first channel being configured to receive the expandable containment device for deployment and the second channel being configured to receive the delivery device for deployment.

2. The device according to claim 1, wherein:
the expandable member has a first surface, a second surface, and a fillable cavity disposed there between; and
the first surface and the second surface have complimentary convex and/or concave shapes.

3. The device according to claim 2, wherein when the expandable member is in the expanded state, the expandable member is configured to engage an exterior surface of the bone.

4. The device according to claim 3, wherein the delivery device is configured to deliver the therapeutic material to the treatment area when the expandable member of the containment device is in the expanded state.

5. The device according to claim 1, wherein the first channel has a diameter that is smaller than a diameter of the second channel.

6. The device according to claim 5, further comprising:
another access device having a first end, a second end, and a length there between;
the other access device including a channel disposed along the length from the first end to the second end; and
the other access device being configured to receive one or more boring devices.

7. The device according to claim 6, wherein:
the access device has a diameter along the length;
the other access device has a diameter along the length;
the diameter of the other access device is larger than a diameter of the access device; and
the channel of the other access device is configured to receive the other access device.

8. The device according to claim 7, wherein each of the access device and the other access device includes a handle member disposed about the second end, the handle members being configured to mate when the access device is fully deployed within the other access device.

9. The device according to claim 6, wherein:
the other access device has a diameter at the first end that is larger than a diameter at the second end, the diameter of the other access device along the length being tapered.

10. The device according to claim 1, further comprising:
one or more boring devices, each boring device including a first end, a second end, and a length there between;
each boring device including a cutting edge disposed at the first end; and
the one or more boring devices being configured to form a path through the bone cavity from a first surface to a second surface of the bone.

11. A method of stabilizing a bone by delivering a therapeutic material comprising:
deploying an access device into an access path within a bone cavity of a bone from a first surface of the bone, the access device having a first end, a second end, and a length;
the access device including a plurality of channels extending along the length from the first end to the second end;
deploying a containment device having an expandable member in a collapsed state through one of the channels of the access device and through the access path until the expandable member is outside of a second surface of the bone, the first surface and the second surface of the bone being disposed on opposing ends of the access path;
expanding the expandable member so that the expandable member engages the second surface of the bone and covers an opening of the access path;
deploying a delivery device through another one of the channels of the access device; and
delivering a therapeutic material to a treatment area while the expandable member is expanded.

12. The method according to claim 11, wherein:
the expandable member has a first surface, a second surface, and a fillable cavity disposed there between; and
the first surface and the second surface have complimentary convex and/or concave shapes.

13. The method according to claim 11, wherein the channel has a diameter that is smaller than a diameter of the other channel.

14. The method according to claim 13, further comprising:
deploying another access device with a boring device mated with the other access device to form a first portion of the access path from the first surface of the bone to the treatment area;
removing the boring device; and
deploying the access device into the other access device;
wherein the access device has a diameter that is smaller than a diameter of the other access device.

15. The method according to claim 14, further comprising:
deploying another boring device through the access device to form a second portion of the access path from the treatment area to the second surface of the bone.

16. The method according to claim 13, further comprising:
deploying another access device with a boring device mated with the other access device to form a portion of the access path from the first surface of the bone to the treatment area;
removing the boring device;
deploying another boring device into the other access device,
removing the other access device; and
deploying the access device over the other boring device.

17. The method according to claim 16, the other access device has a diameter at the first end that is larger than a diameter at the second end, the diameter of the other access device along the length being tapered.

18. The method according to claim 15, wherein each of the boring device and the other boring device includes a drill tip, the drill tip of the boring device and the other boring device being different.

19. The method according to claim 16, further comprising:
collapsing the expandable member and removing the containment device from the bone cavity through the access device;
removing the delivery device through the other channel from the bone cavity; and
removing the access device from the bone cavity.

20. The method according to claim 11, wherein the therapeutic material is a bone filling material.
